(19) United States
(12) Patent Application Publication
(10) Pub. No.: US 2008/0280999 A1
(43) Pub. Date: Nov. 13, 2008

(54) Process for Making Pharmaceutical Compositions with a Transient Plasticizer

(75) Inventor: Jay Parthiban Lakshman, Cedar Knolls, NJ (US)

Correspondence Address: NOVARTIS;CORPORATE INTELLECTUAL PROPERTY, ONE HEALTH PLAZA 104/3, EAST HANOVER, NJ, 07936-1080, US

(73) Assignee: NOVARTIS AG, Basel (CH)

(21) Appl. No.: 12/091,206

(22) PCT Filed: Nov. 7, 2006
(86) PCT No.: PCT/US06/60594

§ 371 Date: Apr. 23, 2008

(30) Foreign Application Priority Data
Nov. 9, 2005 (US) 60/735094

(51) Int. Cl. A61K 47/34

(52) U.S. Cl. 514/772.3

(57) ABSTRACT

A process for making a solid oral dosage form that has a therapeutic compound (e.g., a poorly soluble and/or poorly compactible therapeutic compound) and a polymer. The process is accomplished by the use of an extruder. A transient plasticizer, e.g., a liquefied gas such as supercritical carbon dioxide, is added to facilitate processing of the materials. The transient plasticizer can serve to lower the viscosity of the mixture being processes and/or enhance the solubility of the therapeutic compound.

### FIELD OF THE INVENTION

The present invention relates to a process for making solid oral dosage forms of a therapeutic compound, e.g., a poorly soluble therapeutic compound or a poorly compactible compound. The process features the use of a transient plasticizer in a extruder, e.g., a twin-screw extruder.

### BACKGROUND OF THE INVENTION

Poorly soluble therapeutic compounds typically have low absorption and poor bioavailability. To enhance their dissolution rates and solubility, researchers have sought to reduce the particle size of the therapeutic compounds thereby increasing the surface area available for dissolutions. One type of dosage form used to accomplish this particle size reduction is a solid dispersion. Solid dispersions can be characterized as a molecular dispersion of the therapeutic compound in an inert carrier in a solid state.

Various methods have been used to achieve a solid dispersion. For example, an eutectic mixture of the therapeutic compound and the carrier, e.g., a polymer, can be made by melting their physical mixture. The drawback of this approach is that therapeutic compound begins to decompose due to the high temperatures needed to melt the components.

Another technique, the solvent method, proceeds with dissolving the therapeutic compound and carrier in a solvent, such as an organic solvent, to form a uniform solution and subsequently evaporating the solvent. This technique may not be desirable because a residual level of the organic solvent may still be present in the finished solid dispersions. Additionally, organic solvents are undesirable because of environmental and/or economic considerations.

Poorly compactible therapeutic compounds typically do not form physically integral compacts in of themselves that can withstand ordinary handling. To improve the robustness and manufacturability of such tablets formulators typically use significant amounts of excipients blended-in with the therapeutic compound prior to compaction. These blends are wet granulated with binders in order to maximize the loading of the therapeutic compound. With optimized formulations and processes, formulations can be made to bear therapeutic compound loads as high as 60%. However, it is usually difficult to achieve loads of 70% or higher but it is much more difficult to have drug loads of 70-80% or higher.

For compounds that are used in large doses, such as 600 mg and 1000 mg, tablet size and swallowing size may become issues when large amounts of excipients are used in the formulations. Similarly, some therapeutic compounds may become unstable when large amounts of excipients are used. Thus, minimizing the amount of excipients can lead to better stability and longer shelf-life. Additionally, costs may be reduced with lesser amounts of excipients.

Thus, there is a need for a method of preparing pharmaceutical compositions, especially solid dispersions, of poorly soluble therapeutic compounds without the risk of thermal decomposition of the therapeutic compound and/or the need for the use of an organic solvent. Similarly, for poorly compactible therapeutic compounds, where minimal amounts of polymer are available, melt viscosities can be very high making it difficult to process. This invention addresses that need by the use of a transient plasticizer during the processing of a therapeutic compound and a carrier in a extruder.

### SUMMARY OF THE INVENTION

The present invention relates to marking a pharmaceutical composition that includes the following steps:
(a) combining at least a therapeutic compound (e.g., a poorly soluble and/or poorly compactible therapeutic compound) and a polymer in an extruder, such as a twin screw extruder;
(b) heating either the therapeutic compound and/or polymer forming a mixture by heating;
(c) introducing a transient plasticizer into the mixture to form a plasticized mixture; the transient plasticizer can be a liquefied gas, such as a supercritical fluid.

Particularly useful is supercritical carbon dioxide;
(d) removing the transient plasticizer from the plasticized mixture to form a product; and
(e) cooling the product.

In an alternative embodiment, the transient plasticizer can be introduced into the extruder equipment simultaneously with the introduction of the therapeutic compound and polymer.

In yet another embodiment, a partially transient plasticizer can be substituted for the transient plasticizer. For example, sorbitol hydrate can be used as a partially transient plasticizer. The water can be removed from the sorbitol hydrate leaving the sorbitol.

### DETAILED DESCRIPTION OF THE INVENTION

The present invention relates to a process for preparing pharmaceutical compositions containing a therapeutic compound, especially a poorly soluble or a poorly compactible therapeutic compound. The inventive process features processing of a therapeutic compound, a polymer (e.g., a hydrophilic polymer) and a transient plasticizer in a extruder.

As used herein the term “pharmaceutical composition” means a mixture or dispersion containing a therapeutic compound to be administered to a mammal, e.g., a human in order to prevent, treat or control a particular disease or condition affecting the mammal. A pharmaceutical composition itself can refer to a solid dispersion (e.g., an entire tablet) or be composed of components each in of itself being a solid dispersion (e.g., granules that are subsequently compacted into tablets).

As used herein the term “pharmaceutically acceptable” refers to those compounds, materials, compositions and/or dosage forms, which are, within the scope of sound medical judgment, suitable for contact with the tissues of mammals, especially humans, without excessive toxicity, irritation, allergic response and other problem complications commensurate with a reasonable benefit/risk ratio.

As used herein the term “therapeutic compound” means any compound, substance, drug, medicament, or active ingredient having a therapeutic or pharmacological effect, and which is suitable for administration to a mammal, e.g., a human, in a composition that is particularly suitable for oral administration.

Therapeutic compounds that are particularly suited for the present invention are those that are poorly soluble or insoluble in water. As used herein, the term “poorly water-soluble” or “poorly soluble” refers to having a solubility in water at 20° C. of less than 1%, i.e., a “sparingly soluble to practically insoluble, or insoluble drug” as described in Remington, The Science and Practice of Pharmacy, 21^{st} Edition, p. 212, D. B. Troy, Ed., Lippincott Williams & Wilkins (2005).

Also useful in the present invention are those that are poorly compactible. As used herein, the term “poorly compactible” refers to a compound that does not easily bond to form a tablet upon the application of a force. A tablet produced solely of the therapeutic compound weighing 1 g and compressed under a force ranging from 5-25 kN with a dwell time under 30 seconds, would provide friability at or above an acceptable limit of 1.0% (w/w) when tablets weighing approximately 10 g (or at least 20 units) are tested after 500 drops immediately after compression. Such compounds may require additional processing and special formulating, e.g., wet granulating or roller compacting, prior to compression. High dosages of a therapeutic compound may also render a therapeutic compound not appropriate for direct compression because of poor flowability and poor compressibility.

Examples of therapeutic classes of therapeutic compounds include, but are not limited to, anti-inflammatory substances, coronary dilators, cerebral dilators, peripheral vasodilators, anti-infectives, psychotropics, antimanics, stimulants, antihistamines, anti-cancer therapeutic compounds, gastrointestinal sedatives, anti-anginal therapeutic compounds, vasodilators, antiarrythmics, anti-hypertensive therapeutic compounds, vasoconstrictors and migraine treatments, anticoagulants and antithrombotic therapeutic compounds, analgesics, anti-pyretics, hypnotics, anti-nauseants, anti-convulsants, neuromuscular therapeutic compounds, hyper- and hypoglycemic agents, thyroid and anti-thyroid preparations, diuretics, anti-spasmodics, uterine relaxants, anti-obesity therapeutic compounds, anabolic therapeutic compounds and erythropoietic therapeutic compounds.

Exemplary poorly soluble therapeutic compounds include, but are not limited to, ibuprofen, indomethacin, nifedipine, phenacetin, phenyloin, digitoxin, digoxin, nilvadipine, diazepam, griseofulvin, chloramphenicol and sulfathiazole.

Exemplary poorly compactible therapeutic compounds include, but are not limited to, acetaminophen, ibuprofen and phenacetin.

The therapeutic compound(s) is present in the pharmaceutical compositions of the present invention in a therapeutically effective amount or concentration. Such a therapeutically effective amount or concentration is known to one of ordinary skill in the art as the amount or concentration varies with the therapeutic compound being used and the indication which is being addressed. For example, in accordance with the present invention, the therapeutic compound may be present in an amount by weight of about 0.05% to about 99% weight of pharmaceutical composition. In one embodiment, the therapeutic compound may be present in an amount by weight of about 10% to about 95% by weight of the pharmaceutical composition.

As used herein, the term “polymer” refers to a polymer or mixture of polymers that have a glass transition temperature, softening temperature or melting temperature by itself or in combination. The glass transition temperature (“Tg”) is the temperature at which such polymer's characteristics change from that of highly viscous to that of relatively less viscous mass. Types of polymers include, but are not limited to, water-soluble, water-swellable, water-insoluble polymers and combinations of the foregoing. Particularly useful for poorly soluble compounds in the present invention are hydrophilic polymers which would be those that are water-soluble and/or water-swellable. For a poorly compactible compound, any type of polymer as specified above is suitable. For a highly water-soluble therapeutic compound, a water-insoluble polymer may be necessary.

When the polymer is blended with a poorly soluble therapeutic compound, using a twin screw hot melt extruder, the glass transition temperature (“T′g”) of the blend may be modulated/increased for better stabilizing the amorphous drug from recrystallization will have a lowered T′g.

Examples of polymers include, but are not limited to:
homopolymers and copolymers of N-vinyl lactams, e.g., homopolymers and copolymers of N-vinyl pyrrolidone (e.g., polyvinylpyrrolidone), copolymers of N-vinyl pyrrolidone and vinyl acetate or vinyl propionate;
cellulose esters and cellulose ethers (e.g., methylcellulose and ethylcellulose) hydroxyalkylcelluloses (e.g., hydroxypropylcellulose), hydroxyalkylalkylcelluloses (e.g., hydroxypropylmethylcellulose), cellulose phthalates (e.g., cellulose acetate phthalate and hydroxylpropylmethylcellulose phthalate) and cellulose succinates (e.g., hydroxypropylmethylcellulose succinate or hydroxypropylmethylcellulose acetate succinate);
high molecular polyalkylene oxides, such as polyethylene oxide and polypropylene oxide and copolymers of ethylene oxide and propylene oxide;
polyacrylates and polymethacrylates (e.g., methacrylic acid/ethyl acrylate copolymers, methacrylic acid/methyl methacrylate copolymers, butyl methacrylate/2-dimethylaminoethyl methacrylate copolymers, poly(hydroxyalkyl acrylates), poly(hydroxyalkyl methacrylates));
polyacrylamides;
vinyl acetate polymers, such as copolymers of vinyl acetate and crotonic acid, partially hydrolyzed polyvinyl acetate;
polyvinyl alcohol; and
oligo- and poly-saccharides, such as carrageenans, galactomannans and xanthan gum, or mixtures of one or more thereof.

As used herein, the term “plasticizer” refers to a material that may be incorporated into the pharmaceutical composition in order to decrease the glass transition temperature and the melt viscosity of a polymer by increasing the free volume between polymer chains. Plasticizers, e.g., include, but are not limited to, water; citrate esters, (e.g., triethylcitrate, triacetin); low molecular weight poly(alkylene oxides) (e.g., poly(ethylene glycols), poly(propylene glycols), poly(ethylene/propylene glycols)); glycerol, pentaerythritol, glycerol monoacetate, diacetate or triacetate; propylene glycol; sodium diethyl sulfosuccinate; and the therapeutic compound itself. The plasticizer can be present in concentration from about 0-25%, e.g., 0.5-15%, e.g., 1-20% by weight of the pharmaceutical composition. Examples of plasticizers can also be found in The Handbook of Pharmaceutical Additives, Ash et al., Gower Publishing (2000).

As used herein, the term “transient plasticizer” refers to any material or substance that is used in the process of melt extrusion or melt granulation, wherein all or part of that material or substance is removed during or subsequently after melt extrusion or melt granulation, e.g., water, organic or inorganic hydrates, liquefied gases, pressurized gases or supercritical fluids. Partial removal refers to the removal of a portion of the transient plasticizer. For example, if a hydrate is used, the water fraction of the transient plasticizer might only be removed leaving the balance of the compound. For example, if sorbitol hydrate were used as a transient plasticizer, then only water from the hydrate is removed leaving the sorbitol.

The transient plasticizer can serve to facilitate dissolution of the therapeutic compound in the polymer and/or function as a processing aid to reduce the viscosity of the therapeutic compound and polymer mixture.

As used herein, the term “liquefied gas” refers to a gas (which typically exists in a gaseous state at room temperature and pressure) that is compressed or pressurized into a liquid. Examples of liquefied gases include, but are not limited to, supercritical fluids, nitrogen, nitrous oxide, ethane, propane, ammonia and hydrofluorocarbons.

As used herein, the term “supercritical fluid” refers to a fluid at or above its critical pressure (P_{c}) and critical temperature (T_{c}) simultaneously. Thus, a fluid above its P_{c} and at its T_{c} is in a supercritical state. A fluid at its critical pressure and above its T_{c} is also supercritical. As used herein, supercritical fluids also encompass both near supercritical fluids and subcritical fluids. A “near supercritical fluid” is above but close to its P_{c} and T_{c} simultaneously. A “subcritical fluid” is above its P_{c} and close to its T_{c}.

Examples materials that can be compressed into a supercritical fluid include, but are not limited to, carbon dioxide, methane, benzene, methanol, ethane, ethylene, xenon, nitrous oxide, fluoroform, dimethyl ether, propane, n-butane, isobutane, n-pentane, isopropanol, methanol, toluene, propylene, chlorotrifluoro-methane, sulfur hexafluoride, bromotrifluoromethane, chlorodifluoromethane, hexafluoroethane, carbon tetrafluoride, decalin, cyclohexane, xylene, tetralin, aniline, acetylene, monofluoromethane, 1,1-difluoroethylene, ammonia, water, nitrogen and mixtures thereof. Particularly useful is carbon dioxide which has a T_{c} of 31.1° C. and a P_{c} of 7.38 MPa.

Particularly useful in the present invention is, e.g., a transient plasticizer in which both the hydrophilic polymer and/or therapeutic compound are miscible or partially miscible with. The transient plasticizer helps to dissolve either the therapeutic compound or the polymer.

The transient plasticizer lowers the initial T′g of the therapeutic compound-polymer blend such that it permits processing in a extruder resulting in a lowered Tg (“T″g”); however, after extrusion and distillation of the transient plasticizer, the T″g returns to T′g. This return to T′g helps to prevent recrystallization of the therapeutic compound, e.g., a poorly soluble therapeutic compound.

Although processing a material at a low temperature has a tendency to increase the viscosity of the material, it is expected that a transient plasticizer by virtue of its high diffusivity will lower the viscosity of a material thereby countering and overall overwhelming any viscosity increases attributable to low temperature.

As used herein, the term “melt extrusion” refers to the following compounding process that comprises the steps of:
(a) forming a mixture of a therapeutic compound with a polymer (e.g., separately or simultaneously);
(b) granulating the mixture using an extruder having multiple sections;
(c) introducing a transient plasticizer into the mixture;
(d) optionally heating the mixture while continuing to mix the mixture within the extruder;
(e) removing the transient plasticizer; and
(f) optionally extruding the mixture through a die.

The blending of the therapeutic compound, polymer and transient plasticizer to form an extrudate is accomplished by the use of an extruder. The extrudate, e.g., can serve as an internal phase of granules that is subsequently combined with other pharmaceutically acceptable excipients and compressed to form a solid oral dosage form, e.g., a tablet.

In general, an extruder includes a rotating screw(s) within a stationary barrel with an optional die located at one end of the barrel. Types of extruders particularly useful in the present invention are single-, twin- and multi-screw extruders, optionally configured with kneading paddles. Along the entire length of the screw, distributive kneading of the materials (e.g., the therapeutic compound, polymer, and any other needed excipients) is provided by the rotation and/or counter rotation of the screw(s) within the barrel. Conceptually, the extruder can be divided into at least three sections or barrel zones: a feeding section; a blending section; and a metering section. Any section can further be subdivided into multiple sections.

In the feeding section, the raw materials are fed into the extruder, e.g., from a hopper. The raw materials are then conveyed by transfer elements into the blending section. In the blending section, the raw materials are mixed and/or kneaded by screws and/or paddles attached thereto.

The blending section, itself, can be divided into smaller segments. At the inlet of least one blending segment is, e.g., a dynamic seal(s). In this section of the blending section, the transient plasticizer can be introduced (e.g., if the supercritical fluid is carbon dioxide, it can be introduced as dry ice). This dynamic seal prevents the transient plasticizer from passing back into a prior blending section or the feeding section. Additionally, the dynamic seal(s) allows materials to be fed into the blending section while maintaining the requisite pressures necessary to prevent any transient plasticizer from escaping as a gas.

The plasticized mixture can then be passed into another blending segment for additional mixing (e.g., high shear or distributive mixing). After the blending section is a metering section in which the mixed materials are extruded through an optional die into a particular shape, e.g., granules or noodles. The transient plasticizer can be removed from the mixture when the mixture is extruded from the die.

Alternatively, at any point after plasticizing, a vent can be incorporated into the extruder to allow for the transient plasticizer to escape. For example, a ventport attached to a vacuum line can be used. Also, the pitch or design of the screw elements can be altered such that the escape of transient plasticizer can be controlled.

In another exemplary configuration, the different flights along the length of the screw elements can be used in order to create areas of high and low pressure. For example, if the flights are spaced closely together than pressure is increased, thereby helping to maintain the transient plasticizer. If the flights are sparsely spaced, then low pressure is created to facilitate venting of the transient plasticizer.

Once the granules are obtained, the granules may be formulated into oral forms, e.g., solid oral dosage forms, such as tablets, pills, lozenges, caplets, capsules or sachets, by adding additional conventional excipients which comprise an external phase of the pharmaceutical composition. Examples of such excipients include, but are not limited to, release retardants, plasticizers, disintegrants, binders, lubricants, glidants, stabilizers, fillers and diluents. One of ordinary skill in the art may select one or more of the aforementioned excipients with respect to the particular desired properties of the solid oral dosage form by routine experimentation and without any undue burden. The amount of each excipient used may vary within ranges conventional in the art. The following references which are all hereby incorporated by reference discloses techniques and excipients used to formulate oral dosage forms. See The Handbook of Pharmaceutical Excipients, 4^{th} edition, Rowe et al., Eds., American Pharmaceuticals Association (2003); Remington: the Science and Practice of Pharmacy, 20^{th} edition, Gennaro, Ed., Lippincott Williams & Wilkins (2003); and The Theory and Practice of Industrial Pharmacy, Lachman, Lieberman and Kanig, Eds., 3^{rd} Edition (1986).

Examples of pharmaceutically acceptable disintegrants include, but are not limited to, starches; clays; celluloses; alginates; gums; cross-linked polymers, e.g., cross-linked polyvinyl pyrrolidone or crospovidone, e.g., POLYPLASDONE XL from International Specialty Products (Wayne, N.J.); cross-linked sodium carboxymethylcellulose or croscarmellose sodium, e.g., AC-DI-SOL from FMC; and cross-linked calcium carboxymethylcellulose; soy polysaccharides; and guar gum. The disintegrant may be present in an amount from about 0% to about 10% by weight of the composition. In one embodiment, the disintegrant is present in an amount from about 0.1% to about 1.5% by weight of composition.

Examples of pharmaceutically acceptable binders include, but are not limited to, starches; celluloses and derivatives thereof, for example, microcrystalline cellulose, e.g., AVICEL PH from FMC (Philadelphia, Pa.), hydroxypropyl cellulose hydroxylethyl cellulose and hydroxylpropylmethyl cellulose METHOCEL from Dow Chemical Corp. (Midland, Mich.); sucrose; dextrose; corn syrup; polysaccharides; and gelatin. The binder may be present in an amount from about 0% to about 50%, e.g., 10-40% by weight of the composition.

Examples of pharmaceutically acceptable lubricants and pharmaceutically acceptable glidants include, but are not limited to, colloidal silica, magnesium trisilicate, starches, talc, tribasic calcium phosphate, magnesium stearate, aluminum stearate, calcium stearate, magnesium carbonate, magnesium oxide, polyethylene glycol, powdered cellulose and microcrystalline cellulose. The lubricant may be present in an amount from about 0% to about 10% by weight of the composition. In one embodiment, the lubricant may be present in an amount from about 0.1% to about 1.5% by weight of composition. The glidant may be present in an amount from about 0.1% to about 10% by weight.

Examples of pharmaceutically acceptable fillers and pharmaceutically acceptable diluents include, but are not limited to, confectioner's sugar, compressible sugar, dextrates, dextrin, dextrose, lactose, mannitol, microcrystalline cellulose, powdered cellulose, sorbitol, sucrose and talc. The filler and/or diluent, e.g., may be present in an amount from about 15% to about 40% by weight of the composition.

To make pharmaceutical compositions of the present invention, a therapeutic compound and a polymer are blended in a ratio in a range of 99:1 to 1:25 (on a dry weight basis) prior to, or upon addition into the hopper of an extruder. In one exemplary embodiment, this ratio between the therapeutic compound and granulation excipient can be in a range of 97:3 to 60:40 (on a dry weight basis). Yet in another alternative embodiment, the ratio can be in a range of 97:3 to 75:25 (on a dry weight basis). Furthermore a transient plasticizer can range from about 1-75% by weight of the composition; e.g., 2-50%; e.g., 3-30%; e.g., 4-20% and, e.g., 5-15%. The melt extrusion process may combine some or all of the following steps of unit operation in this order shown or any other alternative sequence:
1. feeding and combining a therapeutic compound and at 40° C. to about 80° C., or e.g., 60° C. into an extruder;
2. softening either the polymer and/or therapeutic compound to facilitate miscibility of the two materials in the mixture. As used herein, “softening” includes heating or melting depending on the nature of the material to be heated. For example, if a crystalline material is to be softened, then “softening” includes melting. If an amorphous material is to be softened, then “softening” can refer to a lowering or reduction of the material's viscosity;
3. introducing and incorporating a transient plasticizer to the mixture. The transient plasticizer can be mixed with the polymer and/or therapeutic compound prior to or after softening;
4. mixing the plasticized mixture, e.g., also with kneading should be continued until a desired level of miscibility as known by one of ordinary skill in the art is obtained. For a poorly compactible therapeutic compound, the mixing should be continued until the therapeutic compound is adequately covered by the polymer;
5. removing the transient plasticizer from the plasticized mixture, e.g., by venting;
6. cooling the resulting mixture to room temperature. The cooling can be accomplished by rapid or controlled cooling mechanisms; for poorly soluble compounds where the therapeutic compound is formulated into an amorphous solid dispersion the cooling should be conducted such that crystallization or recrystallization is minimized or reduced; and
7. optionally extruding the combination through a die.

After cooling, the extrudate can be milled and subsequently screened through a sieve. The granules (which constitute the internal phase of the pharmaceutical composition) are then optionally combined with solid oral dosage form excipients (the external phase of the pharmaceutical composition), i.e., fillers, binders, disintegrants, lubricants and etc. The combined mixture may be further blended, e.g., through a V-blender, and subsequently compressed or molded into a tablet, e.g., a monolithic tablet, or encapsulated by a capsule.

The appropriate temperature for heating (softening) the mixture in the melt extruder depends on the nature of the product being formed. For example, for a solid dispersion of a poorly soluble therapeutic compound it may be necessary to melt or dissolve the therapeutic compound into the polymer in order to raise the Tg of the final formula/binary mixture. In this scenario, the temperature of the melt extruder e.g., is higher than the softening and/or melting points of the therapeutic compound and if necessary, the polymer. However, if one of either the therapeutic compound or polymer readily dissolves or becomes miscible in the other, then the melt extrusion temperature can be higher than just one of the melting/softening points of the therapeutic compound and/or the polymer. In other words, for a crystalline poorly soluble therapeutic compound, it may be better to first melt the compound into an amorphous therapeutic compound to enhance miscibility with the polymer. For an amorphous therapeutic compound, it may be necessary to be above the Tg of the compound. Thus, processing temperatures of the melt extruder may not need to exceed both the melting temperature of the therapeutic compound and the polymer.

For example, in the case of a poorly compactible therapeutic compound, the state of the therapeutic compound (i.e., crystalline versus amorphous) does not factor into determining the heating temperature of the melt extruder except when thermal stability of the drug is inherently poor. Accordingly, the melt extruder is heated higher than the melting point or softening point of the polymer but not necessarily also higher than the poorly compactible therapeutic compound.

Once the tablets are obtained, they can be optionally coated with a functional or non-functional coating as known in the art. Examples of coating techniques include, but are not limited to, sugar coating, film coating, microencapsulation and compression coating. Types of coatings include, but are not limited to, enteric coatings, sustained-release coatings, controlled-release coatings.

The utility of all the pharmaceutical compositions of the present invention may be observed in standard clinical tests in, e.g., known indications of drug dosages giving therapeutically effective blood levels of the therapeutic compound; e.g., using dosages in the range of 2.5-1000 mg of therapeutic compound per day for a 75 kg mammal, e.g., adult and in standard animal models.

The present invention provides a method of treatment of a subject suffering from a disease, condition or disorder treatable with a therapeutic compound comprising administering a therapeutically effective amount of a pharmaceutical composition of the present invention to a subject in need of such treatment.

The following examples are illustrative, but do not serve to limit the scope of the invention described herein. The examples are meant only to suggest a method of practicing the present invention.

### EXAMPLE 1

Pimecrolimus is a poorly compactible therapeutic compound and insoluble in water. Pimecrolimus has a melting point of about 165° C. Thirty (30) mg of pimecrolimus and 275 mg of the polymer, i.e., hydroxypropyl methyl cellulose (3 cps) available as KLUCEL EXF from Hercules Chemical Co. (Wilmington, Del.) are combined and blended in a bin blender for about 200 rotations. The powder blend is introduced into the feed section, or hopper, of a twin screw extruder. A suitable twin screw extruder is the PRISM 16 mm pharmaceutical twin screw extruder available from Thermo Electron Corp. (Waltham, Mass.).

Located at the end of the twin screw extruder is a die with a bore of approximately 3 mm. The twin screw extruder is configured with 5 individual barrel zones, or sections, that can be independently adjusted to different parameters. Starting from the hopper to the die, the zones are respectively heated to the following temperatures: 40° C., 110° C., 120° C., 120° C. and 80° C. with the transient plasticizer being introduced in zone 2 and vented in zone 4 as well as at the exit. If, e.g., a non-transient plasticizer is used (e.g., 15 mg of propylene glycol) then the maximum the temperature of the extruder is set at 130-170° C., which would allow for the melting of the therapeutic compound.

The screw speed is set to 75 rpm, but can be as high as 400 rpm, and the volumetric feed rate is adjusted to deliver between about 30-45 g of material per minute. The throughput rate can be adjusted from 4-80 g/min.

In the zone(s) in which no transient plasticizer, i.e., supercritical fluid, is used, the design of the twin screws can involve simple transfer elements throughout the entire length of the screws except for one zone of mixing elements towards the end of the extruder. Alternatively, the design of the twin screws can involve simple transfer elements throughout the entire length of the screws except for two non-adjacent zones, e.g., one at the beginning at one at the end of the extruder such that the two non-adjacent zones represent, e.g., from about 10-20% of the total screw length.

In the zones in which the transient plasticizer is introduced or present, dynamic seal elements prior to the introduction of the transient plasticizer (e.g., supercritical fluid) and prior to the zone in which the transient plasticizer exits (i.e., vented with or without the help of a vacuum) are implemented. The materials being processed within these barrel zones are subjected to pressures of about 1,500-2,500 psi. The transient plasticizer is introduced at a rate of 0.5-1 kg/hr.

Additional dynamic seal elements can be installed in additional zones in which a high melt pressure needs to be maintained. Alternatively, additional zones of mixing elements can be implemented to reduce melt pressure. Mixing elements can be used outside zones of high pressure that have dynamic seal elements to enhance the venting at low melt pressures.

For example, dynamic seal elements can be used between the first and second barrel zones as supercritical fluid is introduced into the second barrel zone. Mixing and/or transfer elements can be used in the third zone prior to dynamic seal elements between the third and fourth zones. The fourth zone can be equipped with mixing elements and vents.

The extrudate, or granules, from the extruder are then cooled to room temperature by allowing them to stand from approximately 15-20 minutes. Alternatively, the extrudate can be or quench cooled with the help of accessories using cold water/refrigerants or liquid nitrogen. The cooled granules, are subsequently sieved through an 18 mesh screen (i.e., a 1 mm screen).

For the external phase, the magnesium stearate is first passed through an 18 mesh. The magnesium stearate is then blended with the obtained granules using a suitable bin blender for approximately 60 rotations. The resulting final blend is compressed into tablets using a conventional rotary tablet press (Manesty Beta Press) using a compression force ranging between 6 kN and 25 kN. The resulting tablets are monolithic and having a hardness ranging from 5-35 kP. Tablets having hardness ranging from 15-35 kP resulted in acceptable friability of less than 1.0% w/w after 500 drops.

### EXAMPLE 2

A therapeutic compound having the following structure:

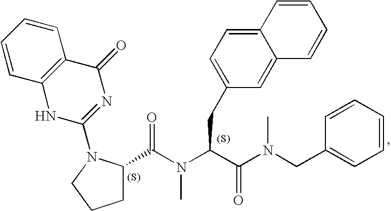

is taken as the therapeutic compound in this example. The melting point of this compound is about 180-182° C. This compound is poorly soluble in water, i.e., 10 mg/L. Fifty (50) mg of this compound and 176 mg of polyvinyl pyrrolidone (K30) are combined and blended in a bin blender for about 200 rotations. The powder blend is introduced into the feed section, or hopper, of a twin screw extruder. A suitable twin screw extruder is the PRISM 16 mm pharmaceutical twin screw extruder available from Thermo Electron Corp. (Waltham, Mass.).

Located at the end of the twin screw extruder is a die with a bore of approximately 3 mm. The twin screw extruder is configured with 5 individual barrel zones, or sections, that can be independently adjusted to different parameters. Starting from the hopper to the die, the zones are respectively heated to the following temperatures: 40° C., 110° C., 130° C., 190° C. and 150° C. The pressure in the zones having the transient plasticizer, supercritical carbon dioxide, is about 1,200-2,000 psi. The supercritical carbon dioxide is introduced at a rate of 0.25-1 kg/hr.

The screw speed is set to 75 rpm, but can be as high as 400 rpm, and the volumetric feed rate is adjusted to deliver between about 30-45 g of material per minute. The throughput rate can be adjusted from 4-80 g/min.

Removal of the supercritical carbon dioxide is accomplished by venting to the atmosphere.

### EXAMPLE 3

Metformin, a poorly compactible compound is taken as the therapeutic compound in this example. The melting point of this compound is about 232° C. A thousand mg of this compound and 99 mg of hydroxylpropyl cellulose are combined and blended in a bin blender for about 200 rotations. The powder blend is introduced into the feed section, or hopper, of a twin screw extruder. A suitable twin screw extruder is the PRISM 16 mm pharmaceutical twin screw extruder available from Thermo Electron Corp. (Waltham, Mass.).

Located at the end of the twin screw extruder is a die with a bore of approximately 3 mm. The twin screw extruder is configured with 5 individual barrel zones, or sections, that can be independently adjusted to different parameters. Starting from the hopper to the die, the zones are respectively heated to the following temperatures: 40° C., 110° C., 130° C., 170° C. and 185° C. The pressure in the zones having the transient plasticizer, supercritical carbon dioxide, is about 1,200-2,000 psi. The supercritical carbon dioxide is introduced at a rate of 0.25-1 kg/hr.

The screw speed is set to 150 rpm, but can be as high as 400 rpm, and the volumetric feed rate is adjusted to deliver between about 30-45 g of material per minute. The throughput rate can be adjusted from 4-80 g/min.

Removal of the supercritical carbon dioxide is accomplished by venting to the atmosphere.

It is understood that while the present invention has been described in conjunction with the detailed description thereof that the foregoing description is intended to illustrate and not limit the scope of the invention, which is defined by the scope of the following claims. Other aspects, advantages and modifications are within the scope of the claims.

What is claimed:

1. A process for making a pharmaceutical composition comprising the steps of:
(a) combining a therapeutic compound and a polymer to form a mixture in an extruder;
(b) heating said mixture;
(c) introducing a transient plasticizer into said mixture to form a plasticized mixture;
(d) removing said transient plasticizer from said plasticized mixture resulting in a product; and
(e) cooling said product to room temperature.

2. The process of claim 1, wherein said therapeutic compound is a poorly compactible therapeutic compound.

3. The process of claim 1, wherein said therapeutic compound is a poorly soluble therapeutic compound.

4. The process of claim 3, wherein said polymer is a hydrophilic polymer.

5. The process of claim 1, wherein said transient plasticizer is a liquefied gas.

6. The process of claim 5, wherein said liquefied gas is a supercritical fluid.

7. The process of claim 6, wherein said supercritical fluid is supercritical carbon dioxide or supercritical nitrogen.

8. The process of claim 1, wherein said extruder is a twin-screw extruder.

9. A method of enhancing the manufacturability of a pharmaceutical composition comprising the step of introducing a transient plasticizer into a mixture being blended by an extruder.

10. The method of claim 9, wherein said extruder is a twin screw extruder.

11. The process of claim 9, wherein said pharmaceutical composition comprises a therapeutic compound and a polymer.

12. The method of claim 9, wherein said transient plasticizer is a liquefied gas.

13. A process for making a pharmaceutical composition comprising the steps of:
(a) combining a therapeutic compound, a transient plasticizer and a polymer to form a mixture in an extruder;
(b) heating said mixture while maintaining sufficient pressure to keep said transient plasticizer in a liquefied state;
(c) removing said transient plasticizer from said mixture resulting in a product; and
(d) cooling said product to room temperature.

14. The process of claim 13, wherein said therapeutic compound is a poorly compactible therapeutic compound.

15. The process of claim 13, wherein said therapeutic compound is a poorly soluble therapeutic compound.

16. The process of claim 13, wherein said transient plasticizer is a liquefied gas.

17. The process of claim 16, wherein said liquefied gas is a supercritical fluid.

18. The process of claim 16, wherein said supercritical fluid is supercritical carbon dioxide or supercritical nitrogen.
